(19) United States
(12) Patent Application Publication
(10) Pub. No.: US 2009/0187166 A1
(43) Pub. Date: Jul. 23, 2009

(54) FLUID CONNECTOR SYSTEM

(76) Inventor: Peter Jeffrey Young, Norfolk (GB)

Correspondence Address: OSTRAGER CHONG FLAHERTY & BROITMAN PC, 570 LEXINGTON AVENUE, FLOOR 17, NEW YORK, NY, 10022-6894, US

(21) Appl. No.: 12/374,349

(22) PCT Filed: Jul. 19, 2007
(86) PCT No.: PCT/GB2007/002743

§ 371 Date: Jan. 19, 2009

(30) Foreign Application Priority Data
Jul. 20, 2006 (GB) 0614452.1

(51) Int. Cl. A61M 25/16; F16L 35/00

(52) U.S. Cl. 604/535; 285/38

(57) ABSTRACT

The invention relates to a male connector for use in a connector system for connecting conduits for fluid flow there between. The system comprises a male connector and a female connector and the connectors are releasably interengageable to form a fluid-tight connection. The male connector is adapted by a barrier (7), e.g. a disc, to connect only to a standard female slip Luer connector. The invention also relates to an infusion set including the male connector.

The invention provides a connector system for connecting conduits for fluid flow therebetween.

In hospitals and medical facilities, the standard method for attaching infusion equipment such as syringes and pumps to catheters and IV tubes and other similar equipment in situ in a patient involves the use of the Luer connector system. Luer connectors consist of male and female connector parts which mutually interengage in a fluid-tight manner, either by a simple interference fit, known as a slip or, in a variation, by a simple thread, known as a lock.

Intravenous and other medical connectors such as epidural connectors are commonly Luer-locking connections. The male Luer connector is normally on the infusion set and the female connector is normally on the catheter accessing a body cavity such as the blood stream. Connection of the two therefore allows passage of fluid from the infusion set to the patient. Tracheal tube cuff air connectors are commonly Luer slip connectors. It is important that the tubing which carries gas to a tracheal tube cuff is not accidentally connected to an intravenous or epidural connector.

It is an object of the invention to provide a male connector which will connect to a tracheal tube cuff air connector, but not to an intravenous or epidural connector.

According to a first aspect of the invention, there is provided a male connector for use in a connector system for connecting conduits for fluid flow therebetweeen, the system comprising a male connector and a female connector, the connectors being releasably interengageable to form a fluid-tight connection, the male connector being adapted to connect only to a standard female slip Luer connector.

The male connector may comprise means for preventing connection with a standard female locking Luer connector.

The means for preventing connection may comprise a barrier.

The male connector may comprise a first conduit held within a sleeve, the first conduit being insertable in a second conduit provided on the female connector to form a connection between the two conduits, the barrier being connected to or part of the sleeve, and being positioned at the entrance or within the sleeve.

The barrier may be such that at least part of the sleeve has an internal cross-section small enough to prevent a standard female locking Luer connector from being fully inserted in the sleeve with the first conduit of the male connector inserted in the second conduit of the standard female locking Luer connector.

The barrier may comprise a ridge around all or part of the inner circumference of the sleeve.

The barrier may be part of the sleeve, and the shape of the sleeve may be such that a standard female locking Luer connector is prevented from being fully inserted in the sleeve with the first conduit of the male connector inserted in the second conduit of the standard female locking Luer connector.

The barrier may be such that the sleeve has an internal cross-section large enough to allow a standard female slip Luer connector to be fully inserted in the sleeve with the first conduit of the male connector inserted in the second conduit of the standard female slip Luer connector.

The male connector may comprise means to guide the each female connector on insertion of the first conduit into the second conduit.

The guide means may be tapered towards the entrance of the sleeve.

The guide means may comprise the barrier.

According to a second aspect of the invention, there is provided an infusion set, including a male connector as defined above.

The invention will now be illustrated by way of example with reference to the following drawings, of which:

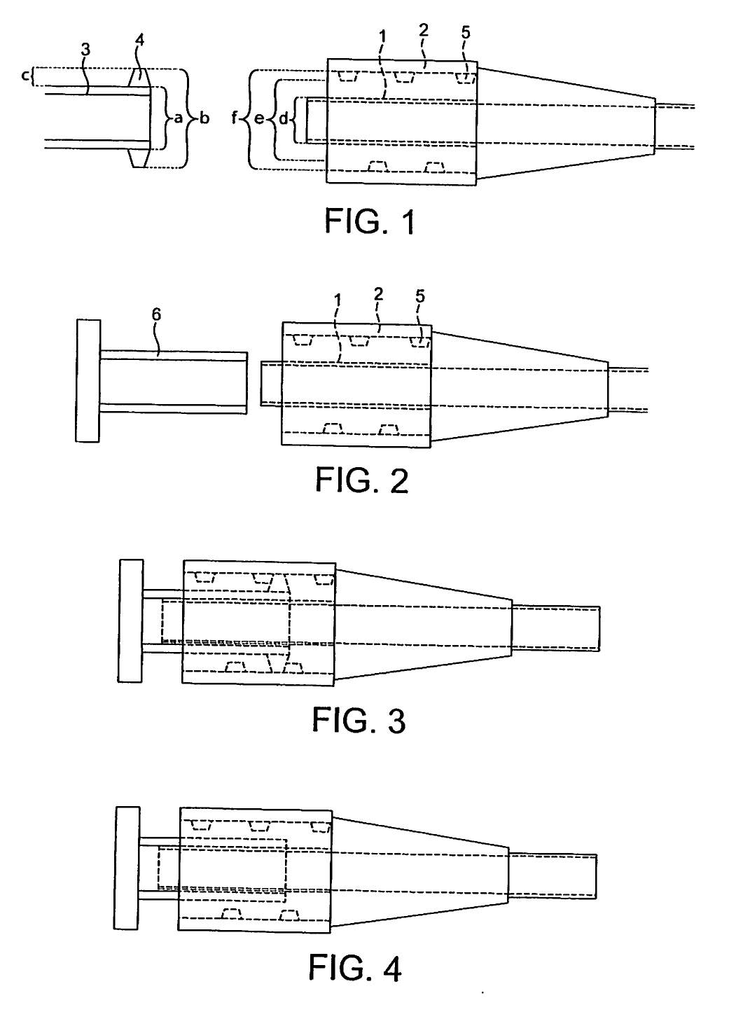

FIG. 1 shows a prior art standard female locking Luer connector and a prior art standard male locking Luer connector;

FIG. 2 shows a prior art standard female slip Luer connector and a prior art standard male locking Luer connector;

FIG. 3 shows the connectors of FIG. 1 linked;

FIG. 4 shows the connectors of FIG. 2 linked;

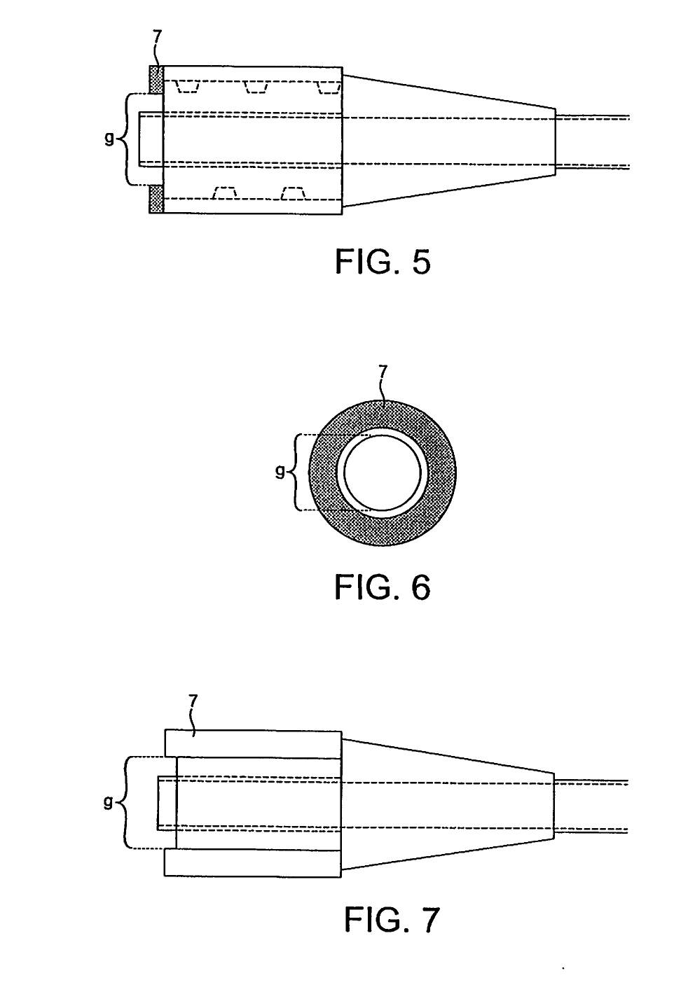

FIG. 5 shows side view of a first embodiment of a male connector according to the invention;

FIG. 6 shows an end view of the male connector shown in FIG. 5;

FIG. 7 shows a second embodiment of a male connector according to the invention;

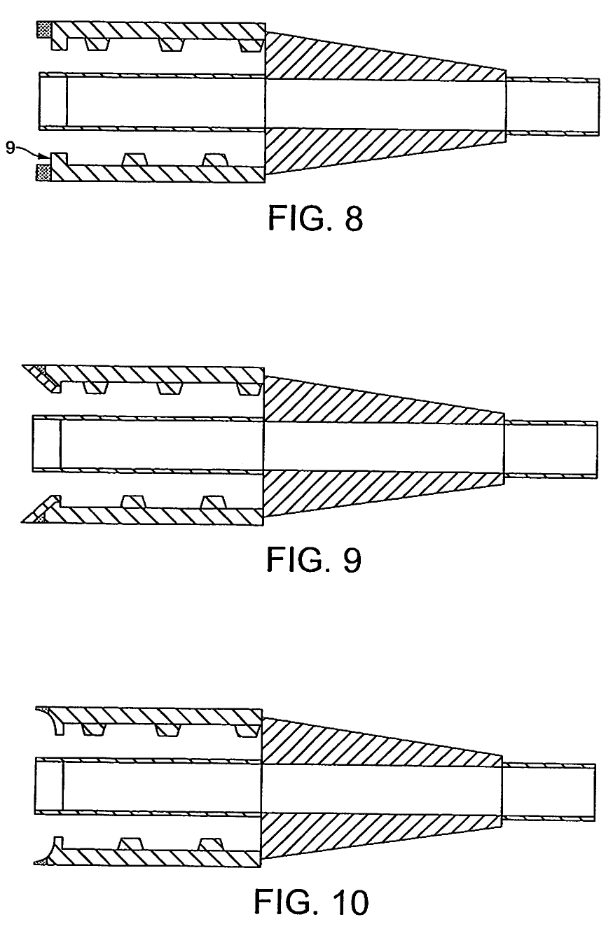

FIG. 8 shows a first modification of the male connectors shown in FIGS. 5 to 7;

FIG. 9 shows a second modification of the male connectors shown in FIGS. 5 to 7; and

FIG. 10 shows a third modification of the male connectors shown in FIGS. 5 to 7;

The standard male Luer locking connector shown in FIG. 1 has a first conduit (1) held within a sleeve (2). The standard female Luer locking connector shown in FIG. 1 has a second conduit (3). The outside of the second conduit (3) carries a number of protuberances (4). The inside of the sleeve (2) carries a screw thread (5). In use, the first conduit (1) is inserted in the second conduit (3) to form a fluid-tight connection between the two connectors, and the protuberances (4) of the standard female Luer connector engage the screw thread (5) of the standard male Luer connector (2) as shown in FIG. 3.

The standard male Luer locking connector shown in FIG. 2 also has a first conduit (1) held within a sleeve (2). The standard female Luer slip connector shown in FIG. 2 has a second conduit (6), but no protuberances. In use, the first conduit (1) is inserted in the second conduit (6) to form a fluid-tight connection between the two connectors as shown in FIG. 4.

The dimensions a-f of current standard Luer connectors are indicated in European standard EN1707 of January 1997.

FIGS. 5 to 7 show two embodiments of the present invention.

Each embodiment comprises a male Luer connector with an additional disc (7) at the entrance to the sleeve (2). The diameter of the disc (7) is such that dimension g is smaller than dimension b of the standard female Luer lock connector. This means that that the protruberances (4) of the standard female Luer lock connector are unable to enter into the sleeve (2) of the connector of the invention, with the result that thestandard female Luer lock connector is unable to connect with the connector of the invention. However, dimension g is not so small that it prevents connection of the prior art Luer slip connector.

To prevent attachment, dimension g must only be smaller than dimension b for enough of the circumference of the connector to prevent the protruberances (4) of the standard female Luer lock connector from entering into the sleeve (2) of the connector of the invention. Accordingly, there are many other possible arrangements. For example, the disc may extend around only part of the circumference of the sleeve, or the cross-section of the sleeve may be non-cylindrical.

The connector may be coloured to assist the user in identifying that it is not intended for intravenous connection.

The connector may have a clip attached designed to engage in a groove, ridge, notch or protuberance on the outer surface of the female slip connector to secure the connector in place.

The connector may be attached to a syringe. In prior art, a syringe can have a male slip tip end connector or a male luer-locking end connector. The current embodiment has a sleeve which prevents connection to a female luer-locking connector.

The male connector may have a ridge modification or indentation modification at the entrance to the connector. The ridge or indentation helps to guide the female connector into place. This can have perpendicular straight (FIGS. 8 and 9) or curved edges (FIG. 10) or edges with other surface configurations.

Another embodiment of the rim modification comprises radial (FIG. 1) or circumferential (FIG. 12) slopes as shown by the arrows. This sloping helps to improve alignment in embodiments where the barrier does not extend around the entire circumference of the sleeve (2). The circumferential slopes cause a wave profile with nadirs at the gaps (11) and peaks at the midpoints (10).

Another embodiment of the rim modification comprises a combination of both radial and circumferential slopes that can improve alignment.

Another embodiment of the rim modification comprises a radial and/or circumferential slope on the inner surface (FIG. 12). This helps disengagement of the attached connector.

Any individual, combination or all of these embodiments can be used together to improve the alignment of the connector.

1. A male connector for use in a connector system for connecting conduits for fluid flow therebetweeen, the system comprising a male connector and a female connector, the connectors being releasably interengageable to form a fluid-tight connection, the male connector being adapted to connect only to a standard female slip Luer connector.

2. A connector system according to claim 1, wherein the male connector comprises means for preventing connection with a standard female locking Luer connector.

3. A connector system according to claim 2, wherein the means for preventing connection comprises a barrier.

4. A connector system according to claim 3, wherein the male connector comprises a first conduit held within a sleeve, the barrier being connected to or part of the sleeve, and being positioned at the entrance to or within the sleeve.

5. A connector system according to claim 4, wherein the barrier is such that at least part of the sleeve has an internal cross-section small enough to prevent a standard female locking Luer connector from being fully inserted in the sleeve with the first conduit of the male connector inserted in the second conduit of the standard female locking Luer connector.

6. A connector system according to claim 5, wherein the barrier comprises a ridge around all or part of the inner circumference of the sleeve.

7. A connector system according to claim 5, wherein the barrier is part of the sleeve, and the shape of the sleeve is such that a standard female locking Luer connector is prevented from being fully inserted in the sleeve with the first conduit of the male connector inserted in the second conduit of the standard female locking Luer connector.

8. A connector system according to claim 4, wherein the barrier is such that the sleeve has an internal cross-section large enough to allow a standard female slip Luer connector to be fully inserted in the sleeve with the first conduit of the male connector inserted in the second conduit of the standard female slip Luer connector.

9. A connector system according to claim 4, wherein each male connector comprises means to guide the female connector on insertion of the female connector into the sleeve.

10. A connector system according to claim 9, wherein the guide means is tapered towards the entrance of the sleeve.

11. A connector system according to claim 10, wherein the guide means comprises the barrier.

12. (canceled)

13. An infusion set including a male connector according to claim 1.
